(19) United States
(12) Patent Application Publication
(10) Pub. No.: US 2014/0024106 A1
(43) Pub. Date: Jan. 23, 2014

(54) MONITORING OF WOUNDS BY MEASUREMENT OF PROTEASE AND PROTEASE INHIBITOR LEVELS IN WOUND FLUIDS

(71) Applicant: Systagenix Wound Management (US), Inc., Quincy, MA (US)

(72) Inventor: Breda Mary Cullen, Skipton (GB)

(21) Appl. No.: 13/951,661

(22) Filed: Jul. 26, 2013

Related U.S. Application Data
(63) Continuation of application No. 11/575,412, filed on Mar. 16, 2007.

(30) Foreign Application Priority Data
Sep. 17, 2004 (GB) 0420774.2

(51) Int. Cl. C12Q 1/37; C12Q 1/34

(52) U.S. Cl. CPC C12Q 1/37; C12Q 1/34
USPC 435/287.9

(57) ABSTRACT

A diagnostic test apparatus for determining a ratio of: (a) at least one endogenous protease enzyme inhibitor, to (b) at least one endogenous protease enzyme, in a sample of a wound fluid. Suitably the protease enzyme is a neutrophil elastase and the protease enzyme inhibitor is alpha-1-antitrypsin. Also provided are wound treatment systems comprising an apparatus according to the invention and a wound dressing comprising an oxidized cellulose. Also provided are methods of diagnosis, prognosis and treatment of wounds using the apparatus and systems of the invention.

### REFERENCE TO A “SEQUENCE LISTING”

The sequence listing submitted via EFS, in compliance with 37 CFR §1.52(e)(5), is incorporated herein by reference. The sequence listing text file submitted via EFS contains the file “98282-000117_ST25.txt”, created on Jul. 24, 2013, which is 5.18 KB in size.

### BACKGROUND OF THE INVENTION

1. Field of the Invention

The present invention relates to an apparatus for monitoring the status of wounds. The present invention further relates to a wound treatment system incorporating an active wound dressing in combination with the wound monitoring apparatus. The present invention further relates to methods of monitoring and treating wounds.

2. Description of Related Art

WO98/00180 and EP-A-1153622 describe the use of freeze-dried sponges comprising oxidized regenerated cellulose (ORC), optionally admixed with collagen, for the treatment of chronic wounds. Dressings based on oxidized cellulose have been found to give outstanding results in the treatment of chronic wounds, including diabetic ulcers, venous ulcers and decubitis ulcers.

C. N. Rao et al. in the Journal of Investigative Dermatology, vol. 105(4), pages 572-578 (1995) describe the results of analysing chronic and acute wound fluids for elastase, alpha-1-antitrypsin (AAT) and fibronectin. It was found that the elastase level was 10 to 40 times higher in the chronic wound fluid. In contrast, alpha-1-antitrypsin was found to be degraded and non-functional in the chronic wound fluids.

GB-A-2393120 describes the use of wound dressings based on ORC in combination with chitosan for the treatment of chronic wounds. The dressings are shown to reduce the levels of elastase and collagenase in the wound fluids.

US-A-2003/0119073 describes sensors for the assay of catabolic protease enzymes in wound fluid. The analyte enzymes include human neutrophil elastase (hNE). It is suggested that the invention can be used in a method of treating chronic wounds by detecting the presence of catabolic protease enzymes, and then treating the wound with inhibitors that are specific for the detected enzymes.

### BRIEF SUMMARY OF THE INVENTION

It has been found by the present inventor that a sub-group of chronic wound patients exhibit a particularly large improvement in wound healing when treated with collagen/ORC sponges. It is an object of the present invention to provide a means to identify these patients as early as possible so that they can receive maximum benefit from therapy with oxidized cellulose. It is a further object of the invention to avoid unnecessary oxidized cellulose therapy on other patients who may be less likely to benefit.

It has now been found that oxidized cellulose therapy is particularly effective for the treatment of chronic wounds in which the wound fluid contains a high initial level of endogenous protease enzymes. Furthermore, it has been found that patients who show a good clinical response to treatment also exhibit a rapid decrease in endogenous protease enzymes. Finally, it has been found that the ratio of the activities of endogenous protease inhibitors such as alpha-1-antitrypsin to the activities of endogenous protease enzymes such as elastase is a particularly good predictor of the success of treatment with oxidized cellulose therapy.

In a first aspect, the present invention provides a diagnostic test apparatus for determining, a ratio of: (a) at least one endogenous protease inhibitor to (b) at least one endogenous protease enzyme, in a sample of a wound fluid.

The ratio may be the ratio of the free concentrations of the markers (a) and (b) in the wound fluid. In other embodiments, the activity of one or both marker types in the wound fluid may be measured as a proxy for the free concentration thereof. The term “level” is used herein to signify either the free concentration of a marker or its activity.

The term “determining” includes measuring a numerical value of said ratio, or it may consist only of determining if the ratio falls above or below a predetermined threshold ratio.

The term “a wound fluid” refers to any wound exudate or other fluid (suitably substantially not including blood) that is present at the surface of the wound, or that is removed from the wound surface by aspiration, absorption or washing. The measuring is suitably carried out on wound fluid that has been removed from the body of the patient, but can also be performed on wound fluid in situ. The term “wound fluid” does not normally refer to blood or tissue plasma remote from the wound site.

It has been found that the ratio of the endogenous protease inhibitor levels to levels of endogenous proteases in wound fluid, whether before or during treatment with a protease inhibitor dressing such as an oxidized cellulose dressing, correlates to the likelihood of (and rate of) healing by means of this therapy. Without wishing to be bound by any theory, it is thought that very low values of these ratios are due to degraded or otherwise inactive protease inhibitors, such that the patient is unlikely to benefit from oxidized cellulose therapy in such cases. Intermediate values of the ratios are characteristic of patients having elevated protease levels with relatively little degradation of the protease inhibitors. Such patients have been found to benefit from oxidized cellulose therapy. The calculated ratios between the measured concentrations of the different markers may also be useful to eliminate false positives and to correct for different wound fluid concentrations.

Suitably, the endogenous protease is selected from the group consisting of neutrophil proteases and macrophage proteases. Examples of neutrophil/macrophage proteases include neutrophil elastase, matrix metalloproteinases (e.g. MMP-9, MMP-8, MMP-1, MMP-12), proteinase 3, plasmin, low molecular weight gelatinases and latent or active elastases, interleukin converting enzymes and tumor necrosis factor (TNFα) converting enzymes. Suitably, the said at least one protease comprises one or more proteases selected from the group consisting of neutrophil elastase and matrix metalloproteinases. Most suitably, the said at least one protease comprises or consists essentially of elastase, in particular neutrophil elastase.

Suitably, the protease inhibitor is selected from the group consisting of elastinil, elafin, secretory leukocyte proteinase inhibitor, alpha-1-macroglobulin, alpha-1-antitrypsin (AAT), and mixtures thereof. Most suitably, the protease inhibitor is AAT.

It will be appreciated that the concentration of more than one marker of each type may be measured. In certain embodiments, the concentrations of at least two, three or four markers are monitored.

The apparatus in the systems according to the present invention may contain diagnostic test devices specifically adapted for detecting the one or more analyte markers. For example, the apparatus may comprise a first device specifically adapted to measure the level of the protease enzyme, and a second device specifically adapted to measure the level of the inhibitor. Suitably, the apparatus comprises a single device specifically adapted to measure the level of both analytes (protease and protease inhibitor). The term “specifically adapted” herein signifies that the device comprises at least one substance that reacts selectively with the analyte. The substance may for example comprise a selective binding partner such as an immunological binding partner, for the analyte. In other embodiments, the substance may comprise is a specific substrate for the analyte, for example a peptide sequence that is cleaved selectively by an analyte protease enzyme. Suitably, the selective reagent is immobilized in the device, for example by chemical or physical bonding to a solid substrate in said device, as described in more detail below.

As noted above, the diagnostic apparatus according to the present invention may contain one or more selective binding partners to bind the one or more analyte molecules present in the sample. Suitable immunological binding partners include polyclonal antibodies and monoclonal antibodies.

If polyclonal antibodies are desired, a selected mammal, such as a mouse, rabbit, goat or horse, may be immunised with the monitored marker. The monitored marker used to immunise the animal can be obtained by any suitable technique, for example, it can be purified from a wound fluid sample from an infected wound, it can be derived by recombinant DNA technology or it can be synthesized chemically. If desired, the monitored marker can be conjugated to a carrier protein. Commonly used carriers to which the monitored markers may be chemically coupled include bovine serum albumin, thyroglobulin and keyhole limpet haemocyanin. The optionally coupled monitored marker is then used to immunise the animal. Serum from the immunised animal is collected and treated according to known procedures, for example by immunoaffinity chromatography.

Monoclonal antibodies to the monitored marker can also be readily produced by one skilled in the art. The general methodology for making monoclonal antibodies using hybridoma technology is well known.

Panels of monoclonal antibodies produced against the monitored marker can be screened for various properties, i.e., for isotype, epitope, affinity, etc. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions may also be of use. Humanised antibodies may also be used. The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is, an antibody having two different antigen binding domains, each domain being directed against a different epitope.

Phage display technology may be utilised to select genes which encode antibodies with binding activities towards the monitored marker either from repertoires of PCR amplified V-genes of lymphocytes from humans screened for possessing the relevant antibodies, or from naive libraries. The affinity of these antibodies can also be improved by chain shuffling.

Where antibodies generated by the above techniques, whether polyclonal or monoclonal, are employed as reagents in immunoassays, radioimmunoassays (RIA) or enzyme-linked immunosorbent assays (ELISA), the antibodies can be labelled with an analytically-detectable reagent such as a radioisotope, a fluorescent molecule or an enzyme.

As used herein, the term “antibody” refers to intact molecules as well as to fragments thereof, such as Fab, F(ab′)2 and Fv, which are capable of binding to the antigenic determinant in question. Such antibodies thus bind to the monitored marker.

Suitably, the immunological or other binding partners are immobilised on a solid support material, for example by avidin-biotin linking, or dialdehyde derivatization of the support material, followed by cross-linking to a peptide binding partner. The apparatus may further comprise other immunological binding partners and/or reagents or indicator molecules may for example in a solution that is added to the wound fluid sample.

The solid support materials bearing immunological or other binding partners may be used in a range of immunoassays to analyse the presence of the analytes of interest. For example, the support having antibodies or antibody fragments bound thereto may be used in sandwich immunoassay-type analysis. Alternatively, the support may have analog ligands bound to the antibodies, whereby the molecules present in the wound fluid are detected by affinity displacement immunoassay. Various other immunoassays will be apparent to persons skilled in the art.

The analytes of interest comprise protease enzymes that can modify substrates, for example proteins or polypeptides, by cleavage. Such modification of peptide substrates can be detected to determine the presence or absence of the analyte in a sample. Accordingly, in suitable embodiments, the diagnostic apparatus comprises an indicator moiety that is immobilized or inhibited by a chemical moiety, wherein the chemical moiety comprises an exogenous peptide substrate for the protease enzyme, and the exogenous peptide substrate is cleavable by the analyte protease enzyme to release or activate the indicator moiety.

Suitably, the indicator moiety comprises an indicator enzyme, an enzyme cofactor, a dye, a radioactive moiety, a spin label, a luminescent moiety or a fluorophore. Suitably, the indicator moiety comprises an indicator enzyme or a fluorophore. Suitable indicator enzymes may for example be selected from the group consisting of a laccase (CotA enzyme), alkaline phosphatase, p-galactosidase, acetylcholinesterase, green fluorescent proteins, luciferases and horseradish peroxidases. Suitable fluorophores include umbelliferone, fluorescein, fluorescein isothiocyanate, rhodamine, dichlorotriazinylamine fluorescein, dansyl chloride and phycoerythrin. Suitable luminescent moieties include luminol, luciferase, luciferin, and aequorin.

In the devices in which the indicator moiety comprises an enzyme, the device suitably further comprises a substrate that interacts with the indicator enzyme to give a detectable spectrophotometric, colorimetric, fluorimetric, luminescent, electrochemical or radioactive signal.

In certain embodiments, the indicator moiety comprises an indicator enzyme and the chemical moiety inhibits the indicator enzyme by sterically hindering an active site of the indicator enzyme, or by causing the indicator enzyme to fold into an inactive conformation. Alternatively or additionally, the chemical moiety may tether the enzyme to a solid substrate, whereby release from the substrate by action of the analyte protease activates the enzyme and/or allows the enzyme to migrate to a remote substrate location where it reacts with a suitable substrate (which may be immobilized at the remote location) to give a detectable signal. In yet other embodiments, the device comprises two indicator enzyme moieties linked by the chemical moiety, and cleavage of said peptide by the host-derived protease enzyme results in activation of both enzyme moieties.

In other embodiments, the device comprises an indicator enzyme, and a cofactor for the enzyme that is immobilized or inhibited by the chemical moiety, whereby cleavage of peptide releases or activates the cofactor.

In certain embodiments, the indicator moiety is tethered to a solid substrate by said chemical moiety, and is released from said substrate by cleavage of said exogenous peptide substrate by said protease.

One method for detecting the modification of a substrate by an enzyme is to label the substrate with two different dyes, where one dye serves to quench the fluorescence of the other dye by fluorescence resonance energy transfer (FRET) when the dye molecules are in close proximity. A typical acceptor and donor pair for resonance energy transfer consists of 4-[[-(dimethylamino)phenyl ]azo]benzoic acid (DABCYL) and 5-[(2-aminoethylamino]naphthalene sulfonic acid (EDANS). EDANS is excited by illumination with 336 nanometer light, and emits a photon with a wavelength of 490 nanometers. If a DABCYL moiety is located within 2 nanometers of the EDANS, this photon will be efficiently absorbed. DABCYL and EDANS can be attached to opposite ends of a peptide in the diagnostic material used in the systems of the present invention. If the peptide is intact, FRET will be very efficient. If the peptide has been cleaved by an enzyme analyte, the two dyes will no longer be in close proximity and FRET will be inefficient. The cleavage reaction can be followed by observing either a decrease in DABCYL fluorescence or an increase in EDANS fluorescence (loss of quenching).

Another suitable diagnostic material for use in the systems of the present invention comprises a chromogenic dye conjugated to a solid support by a suitable cleavable substrate moiety, such as a peptide. The chromogenic dye will change color when the linker group is cleaved by the enzyme of interest. For example, para-nitrophenyl is colorless when linked to the support, and turns yellow when cleaved. The analyte concentration can be determined by measuring absorbance at 415 nanometers. Other dyes that produce detectable color change upon cleavage are known to those skilled in the art.

In yet another embodiment, the diagnostic material may comprise a colored support having a differently-colored molecule conjugated thereto by a linker moiety that can be cleaved by an enzyme in the sample. Cleavage of the dye from the colored support can thereby result in a color change of the diagnostic material.

The solid support materials used for the above identified assays of enzyme activity and immuno-assays may comprise any suitable natural or synthetic polymer, including insoluble polysaccharides such as cellulose, and synthetic polymers such as polyacrylates. The cleavable cross-linkages where present generally comprise cleavable oligopeptidic sequences or cleavable oligosaccharides, each typically of twenty residues or fewer, for example from 3 to 15 residues.

The sensitivity of the diagnostic material will depend on a number of factors, including the length of the cleavable linker sequences. Steric hindrance may also be reduced by coupling the cleavable oligopeptidic sequence to the polymer by means of an appropriate spacer. Thus, the oligopeptidic sequences may couple the polymers directly (in which case the cross-linkage consists of the oligopeptidic sequence) or by means of an appropriate spacer. Suitable conjugation methods incorporating spacers are described in U.S. Pat. No. 5,770,229.

Particularly preferred chemical systems for use in the devices of the present invention are described in WO03/063693 and WO2005/021780, the entire contents of which are incorporated herein by reference.

In one embodiment, the indicator enzyme is a laccase that has been inhibited by the peptide substrate. Laccase (diphenol oxidase) is a member of the multi-copper oxidase family of enzymes. Generally, these enzymes require oxygen to oxidize phenols, polyphenols aromatic amines, and other non-phenolic substrates by one electron to create a radical species. It is a suitable indicator enzyme in part due to its stability and oxidation properties. The oxidation of species results in an upaired electron which generates a color change. CotA is highly thermostable.

CotA can be used in the apparatus and devices of the present invention by modifying the sequence to generate a proenzyme form. Analysis of the structure of CotA indicates that an extension of suitable length appended onto the N-terminus of CotA can allow an appended inhibitor to be placed in the active site of the enzyme. The extension peptide is selected to be a cleavage target of the analyte protease. This will allow the blocking extension to be cleaved in the presence of the analyte protease. Analysis of the x-ray structure of CotA has shown that the length of the amino acid chain needed to reach the shortest distance around the structure is about 3 nm.

The modified enzymes with the peptide extension block can be prepared and screened for suitability using standard recombinant methods as described in more detail in WO2005/021780.

As already noted, the endogenous proteases to be detected may include elastase. In such embodiments, suitable substrate linkers may include one or more of the oligopeptidic sequences Lys-Gly-Ala-Ala-Ala-Lys-Ala-Ala-Ala- (SEQ ID NO: 1), Ala-Ala-Pro-Val (SEQ ID NO: 2), Ala-Ala-Pro-Leu (SEQ ID NO: 3), Ala-Ala-Pro-Phe (SEQ ID NO: 4), Ala-Ala-Pro-Ala (SEQ ID NO: 5) or Ala-Tyr-Leu-Val (SEQ ID NO: 6).

In certain embodiments, the proteases to be detected may include a matrix metalloproteinase, in particular MMP-2 or MMP-9. In these embodiments, the cleavable linker may comprise the oligopeptidic sequence -Gly-Pro-Y-Gly-Pro-Z- (SEQ ID NO: 7), -Gly-Pro-Leu-Gly-Pro-Z- (SEQ ID NO: 8), -Gly-Pro-Ile-Gly-Pro-Z- (SEQ ID NO: 9), or-Ala-Pro-Gly-Leu-Z- (SEQ ID NO: 10), where Y and Z are amino acids.

In certain embodiments, the proteases to be detected may include a collagenase. In these embodiments, the cleavable linker may comprise the oligopeptidic sequence -Pro-Leu-Gly-Pro-Z-Arg-Z- (SEQ ID NO: 11), -Pro-Leu-Gly-Leu-Leu-Gly-Z- (SEQ ID NO: 12), -Pro-Gln-Gly-Ile-Ala-Gly-Trp- (SEQ ID NO: 13), -Pro-Leu-Gly-Cys-His- (SEQ ID NO: 14), -Pro-Leu-Gly-Leu-Trp-Ala- (SEQ ID NO: 15), -Pro-Leu-Ala-Leu-Trp-Ala-Arg- (SEQ ID NO: 16), or -Pro-Leu-Ala-Tyr-Trp-Ala-Arg- (SEQ ID NO: 17), where Z is an amino acid.

In certain embodiments, the proteases to be detected may include a gelatinase. In these embodiments, the cleavable linker may comprise the oligopeptidic sequence -Pro-Leu-Gly-Met-Trp-Ser-Arg- (SEQ ID NO: 18).

In certain embodiments, the proteases to be detected may include thrombin. In these embodiments, the cleavable linker may comprise the oligopeptidic sequence -Gly-Arg-Gly-Asp- (SEQ ID NO: 19), -Gly-Gly-Arg-, -Gly-Arg-Gly-Asp-Asn-Pro- (SEQ ID NO: 20), -Gly-Arg-Gly-Asp-Ser- (SEQ ID NO: 21), -Gly-Arg-Gly-Asp-Ser-Pro-Lys- (SEQ ID NO: 22),-Gly-Pro-Arg-, -Val-Pro-Arg-, or-Phe-Val-Arg-.

In certain embodiments, the proteases to be detected may include stromelysin. In these embodiments, the cleavable linker may comprise the oligopeptidic sequence -Pro-Tyr-Ala-Tyr-Trp-Met-Arg- (SEQ ID NO: 23).

In certain embodiments, the proteases to be detected may include a kallikrein. The term “a kallikrein” refers to all serine proteases, whose activation is associated with the degradation of kininogen to form kinins, which are implicated in the onset of pain. Suitable peptide sequences for use in cleavable substrates for kallikrein include -Phe-Arg-Ser-Ser-Arg-Gln- (SEQ ID NO: 24) or -Met-Ile-Ser-Leu-Met-Lys-Arg-Pro-Gln- (SEQ ID NO: 25) that can be degraded by kallikrein at Lys-Arg or Arg-Ser bonds.

The polypeptides of the invention also encompass fragments and sequence variants of the polypeptides and nucleic acids described above. Functional variants can contain substitution of similar amino acids that result in no change or an insignificant change in function. Alternatively, such substitutions may positively or negatively affect function to some degree.

In certain embodiments, the device in the apparatus according to the present invention comprises, or consists essentially of a wound dressing, dipstick or swab. Immobilisation of reaction components onto a dipstick, wound mapping sheet or other solid or gel substrate offers the opportunity of performing a more quantitative measurement. For example, in the case of a reaction linked to the generation of a colour the device may be transferred to a spectrometer. Suitable methods of analysis will be apparent to those of skill in the art.

Immobilisation of the reaction components to a small biosensor device will also have the advantage that less of the components (such as enzyme and substrate) are needed. The device will thus be less expensive to manufacture than a dressing that needs to have a large surface area in order to allow the mapping of a large wound area.

Methods for the incorporation of the components of the assay reaction onto a clinical dressing, “dipstick”, sheet or other biosensor are routine in the art. See for example Fagerstam and Karlsson (1994) Immunochemistry, 949-970.

The device may further comprise a reference assay element for determining the total protein content of the sample, so that the measured levels of marker can be normalised to constant total protein level in order to increase accuracy.

In certain embodiments, the device in the apparatus according to the present invention comprises a housing containing one or more reagents and having an inlet provided therein for introduction of the sample. The housing may be at least partially transparent, or may have windows provided therein, for observation of an indicator region that undergoes a color or fluorescence change. In certain embodiments, the device operates on the lateral flow principle. That is to say, said device comprises a housing having an inlet for the sample and side walls defining a fluid lateral flow path extending from the inlet. By “lateral flow”, it is meant liquid flow in which the dissolved or dispersed components of the sample are carried, suitably at substantially equal rates, and with relatively unimpaired flow, laterally through the carrier. Suitably, the fluid flow path contains one or more porous carrier materials. The porous carrier materials are suitably in fluid communication along substantially the whole fluid flow path so as to assist transfer of fluid along the path by capillary action. Suitably, the porous carrier materials are hydrophilic, but suitably they do not themselves absorb water. The porous carrier materials may function as solid substrates for attachment of reagents or indicator moieties. In certain embodiments of the present invention, the device further comprises a control moiety located in a control zone in said in said device, wherein the control moiety can interact with a component of the wound fluid sample to improve the accuracy of the device.

The size and shape of the carrier are not critical and may vary. The carrier defines a lateral flow path. Suitably, the porous carrier is in the form of one or more elongate strips or columns. In certain embodiments, the porous carrier is one or more elongate strips of sheet material, or a plurality of sheets making up in combination an elongate strip. One or more reaction zones and detection zones would then normally be spaced apart along the long axis of the strip. However, in some embodiments the porous carrier could, for example be in other sheet forms, such as a disk. In these cases the reaction zones and detection zones would normally be arranged concentrically around the center of the sheet, with a sample application zone in the center of the sheet. In yet other embodiments, the carrier is formed of carrier beads, for example beads made from any of the materials described above. The beads may suitably be sized from about 1 micrometer to about 1 mm. The beads may be packed into the flow path inside the housing, or may be captured or supported on a suitable porous substrate such as a glass fiber pad.

It will be appreciated that the devices in the apparatus according to the present invention may be adapted to detect more than one marker or other analyte. For example, a single device may be adapted to detect both the protease enzyme and the protease enzyme inhibitor. This can be done by the use of several different reagents in a single reaction zone, or suitably by the provision in a single device of a plurality of lateral flow paths each adapted for detecting a different analyte. In certain embodiments, the plurality of lateral flow paths are defined as separate fluid flow paths in the housing, for example the plurality of lateral flow paths may be radially distributed around a sample receiving port. In some embodiments, the plurality of fluid flow paths are physically separated by the housing. In other embodiments multiple lateral flow paths (lanes) can be defined in a single lateral flow membrane by depositing lines of wax or similar hydrophobic material between the lanes.

The devices in the apparatus according to the present invention may for example be incorporated into a bacterial sensing device of the kind described in copending application GB 0501818.9 filed on 28 Jan. 2005, the entire content of which is incorporated herein by reference.

An absorbent element may suitably be included in the devices of the present invention. The absorbent element is a means for drawing the whole sample through the device by capillary attraction. Generally, the absorbent element will consist of a hydrophilic absorbent material such as a woven or nonwoven textile material, a filter paper or a glass fiber filter.

The device may further comprise at least one filtration element to remove impurities from the sample before the sample undergoes analysis. The filtration device may for example comprise a microporous filtration sheet for removal of cells and other particulate debris from the sample. The filtration device is typically provided upstream of the sample application zone of the fluid flow path, for example in the inlet of the housing or in the housing upstream of the inlet.

In certain embodiments, the devices in the apparatus according to the present invention include a control moiety in a control zone of the device, wherein the control moiety can interact with a component of the wound fluid sample to improve the accuracy of the device. Suitably, the control zone is adapted to reduce false positive or false negative results. A false negative result could arise for various reasons, including (1) the sample is too dilute, or (2) the sample was too small to start with.

In order to address false negative mechanism, the control zone suitably further comprises a reference assay element for determining the total protease content or the total protein content of the sample, that is to say for establishing that the total protease content or the total protein content of the sample is higher than a predetermined minimum. It is possible to indicate the presence of protein by the use of tetrabromophenol blue, which changes from colorless to blue depending on the concentration of protein present. It is also possible to detect glucose (using glucose oxidase), blood (using diisopropyl-benzene dihydro peroxide and tetramethylbenzidine), leukocytes (using ester and diazonium salt). These may all be useful analytes for detection in the control zone for the reduction of false negatives.

In certain embodiments, the apparatus according to the present invention may further comprise one or more components selected from: a color chart for interpreting the output of the diagnostic device, a sampling device for collecting a sample of a biological fluid such as a wound fluid, a wash liquid for carrying a sample of fluid through the device, and a pretreatment solution containing a reagent for pretreatment of the fluid sample.

Where present, the sampling device may comprise a swab or a biopsy punch, for example a shaft having a swab or biopsy punch attached thereto. Suitably, in these embodiments the diagnostic device includes a sample receiving port, and suitably the sample receiving port and the swab or biopsy punch comprise complementary fitting elements whereby the swab or biopsy punch can be secured to the device with the swab or biopsy punch received in the sample receiving port.

In certain embodiments the fitting element on the shaft may be located from 1 mm to about 30 mm from the base of the swab or the biopsy punch. This is consistent with the use of relatively small sample receiving port on the housing of the diagnostic device. The sample receiving port is typically located on an upper surface of the diagnostic device, and it is typically generally in the form of an upwardly projecting tube, open at the top and having the inlet to the fluid flow path located at the bottom of the tube. Suitable swabs, biopsy punches and sample receiving caps are described in detail in copending applications GB0403976.4 and GB0403978.0 both filed on 23 Feb. 2004, the entire contents of which are incorporated herein by reference.

The fitting element on the shaft may a tapered region of the shaft for forming an interference fit with the housing, for example it may appear as a truncated cone that is coaxial with the shaft and tapers towards the first end of the shaft. Or the whole shaft may have a diameter larger than that of the swab or biopsy punch, with a tapered region adjacent to the first end. In any case, the diameter of the tapered region where it engages with the housing is normally greater than the diameter of the swab or biopsy punch, so that the inlet port can enclose the swab or biopsy punch.

In other embodiments, the engagement element may comprise a snap-fitting projection for forming a snap-fit with one or more complementary projections on an inner surface of the housing, or a threaded projection for forming a screw fit with one or more complementary threads on an inner surface of the cap, or a Luer-lock type fitting.

The swab may be any absorbent swab, for example a nonwoven fibrous swab. Typically the diameter of the swab is about 2 to about 5 mm, for example about 3 mm. In certain embodiments, the swab may be formed from a medically acceptable open-celled foam, for example a polyurethane foam, since such foams have high absorbency and can readily be squeezed to expel absorbed fluids. The biopsy punch will typically be a stainless steel cylindrical punch of diameter about 1 mm to about 10 mm, for example about 3 mm to about 8 mm, suitably about 6 mm.

In certain embodiments the shaft is hollow, whereby a fluid can be passed down the shaft from the second end to expel the biological sample from the swab or the biopsy punch into the diagnostic device. This helps to ensure that all of the sample passes through the device, thereby avoiding false negatives. The shaft may comprise a fitting at the second end for attachment of a syringe or other source of the fluid. In certain embodiments, the apparatus may comprise a reservoir of liquid attached to the second end of the shaft, for example a compressible bulb containing the liquid, which can be activated after use of the swab or biopsy punch. Suitable devices of this kind are described, for example in U.S. Pat. No. 5,266,266, the entire content of which is incorporated herein by reference. In other embodiments, the apparatus may comprise a plunger that can be pushed down the hollow bore of the shaft to expel fluid or other specimens from the swab or biopsy punch.

Another advantage of the hollow shaft is that, where the apparatus is a biopsy punch, the biopsy sample can more readily be pushed or blown out of the punch. The biopsy punch apparatus can further comprise a homogenizing tool that can be passed down the hollow shaft to homogenize a tissue sample in the biopsy punch. This step of homogenizing can be followed, if necessary, by passing liquid down the shaft from the second end to expel the homogenized tissue from the biopsy punch into the device for diagnostic analysis.

The swab or biopsy punch may be sterilized, and may be packaged in a microorganism-impermeable container.

In a second aspect, the present invention provides a wound treatment system comprising: a wound dressing comprising an oxidized cellulose, and an apparatus according to the first aspect of the invention.

The term “oxidized cellulose” refers to any material produced by the oxidation of cellulose, for example with dinitrogen tetroxide. Such oxidation converts primary alcohol groups on the saccharide residues to carboxylic acid groups, forming uronic acid residues within the cellulose chain. The oxidation generally does not proceed with complete selectivity, and as a result hydroxyl groups on carbons 2 and 3 are occasionally converted to the keto form. These keto units introduce an alkali labile link, which at pH 7 or higher initiates the decomposition of the polymer via formation of a lactone and sugar ring cleavage. As a result, oxidized cellulose is biodegradable and bioabsorbable under phsyiological conditions.

The preferred oxidized cellulose for practical applications is oxidized regenerated cellulose (ORC) prepared by oxidation of a regenerated cellulose, such as rayon. It has been known for some time that ORC has haemostatic properties. ORC has been available as a haemostatic product called SURGICEL (Registered Trade Mark of Johnson & Johnson Medical, Inc.) since 1950. This product is produced by the oxidation of a knitted rayon material. A modification of porosity, density and knit pattern led to the launch of a second ORC fabric product, INTERCEED (Registered Trade Mark of Johnson & Johnson Medical, Inc.), which was shown to reduce the extent of post-surgical adhesions in abdominal surgery.

The wound dressing in the systems according to the present invention includes a wound contacting material comprising the oxidized cellulose. The term “wound contacting material” encompasses materials that do not contact the wound surface directly, but that contact the wound fluid e.g. through a porous top sheet. The wound contacting material is normally the wound contacting layer of the dressing in use, and may for example be selected from the group consisting of woven, nonwoven and knitted fabrics, freeze-dried sponges and solvent-dried sponges comprising the oxidized cellulose. The wound contacting material may comprise at least 10% of oxidized cellulose, for example at least 20% or at least 30% by weight of oxidized cellulose.

In preferred embodiments of the present invention, the oxidized cellulose in the wound dressing material is complexed with collagen and/or chitosan to form structures of the kind described in WO98/00180, EP-A-1153622 and/or WO-A-2004/026200, the entire contents of which are expressly incorporated herein by reference. For example, the oxidized cellulose may be in the form of milled ORC fibres that are dispersed in a freeze-dried collagen or chitosan sponge. This provides for sustained release of the oxidized cellulose to the wound, together with certain therapeutic and synergistic effects arising from the complexation with collagen. Suitably, the weight ratio of oxidized cellulose to collagen and/or chitosan in the wound contacting material is from about 10:1 to about 1:10, for example from about 70:30 to about 30:70. Suitably, the wound contacting material comprises at least 75% on a dry weight basis of oxidized cellulose, collagen and chitosan, more suitably at least 90% and most suitably it consists essentially of oxidized cellulose, collagen and/or chitosan.

In a third aspect, the present invention provides a method for treating a wound that exudes a wound fluid comprising the steps of:

(a) establishing a level of at least one endogenous protease enzyme or endogenous protease enzyme inhibitor in the wound fluid, at a point in time;

(b) applying a wound dressing comprising oxidized cellulose to the wound;

(c) establishing the level of the at least one endogenous protease enzyme or endogenous protease enzyme inhibitor in the wound fluid, at a subsequent point in time; and

(d) applying a wound dressing comprising oxidized cellulose to the wound if the level of the at least one endogenous protease enzyme or endogenous protease enzyme inhibitor in the wound fluid in step (c) is less than the level established in step (a).

In a fourth aspect, the present invention provides a method for treating a wound that exudes a wound fluid comprising the steps of:

(a) establishing a ratio of the level of an endogenous protease enzyme inhibitor to the level of an endogenous protease enzyme in the wound fluid, at a point in time;

(b) applying a wound dressing comprising oxidized cellulose to the wound if said ratio falls within predetermined range.

In a fifth aspect, the present invention provides a method for treating a wound that exudes a wound fluid comprising the steps of:

(a) establishing a ratio of the level of an endogenous protease enzyme inhibitor to the level of an endogenous protease enzyme in the wound fluid, at a point in time;

(b) applying a wound dressing comprising oxidized cellulose to the wound;

(c) establishing the ratio of the levels of the endogenous protease enzyme inhibitor to the endogenous protease enzyme in the wound fluid, at a subsequent point in time; and

(d) applying a wound dressing comprising oxidized cellulose to the wound if the said ratio established in step (c) is greater than the ratio established in step (a).

Suitably, the wound dressing compositions, endogenous protease enzymes, and enzyme inhibitors useful in the second aspect of the invention are as hereinbefore defined in relation to the first aspect of the invention. Suitably, the step of establishing said ratio is performed by means of an apparatus according to the present invention, by one of the methods hereinbefore described in relation to the first aspect of the invention.

Suitably, the oxidized cellulose dressing comprises oxidized regenerated cellulose. Suitably, the wound dressing further comprises collagen or chitosan. Suitable embodiments of the dressing are as discussed above in relation to the second aspect of the invention.

Any type of wound may be diagnosed for treatment using the apparatus and methods of the present invention. For example, the wound may be an acute wound such as an acute traumatic laceration, perhaps resulting from an intentional operative incision. More usually the wound may be a chronic wound. Suitably, the chronic wound is selected from the group consisting of venous ulcers, pressure sores, decubitis ulcers, diabetic ulcers and chronic ulcers of unknown aetiology.

To allow measurement of concentration of a marker in the wound fluid, a sample of wound fluid must be added to the measurement apparatus. Measurement may either be made in situ, or fluid may be removed from the wound for analysis in the device.

The methods according to the present invention may alternatively comprise an aqueous assay step. Wound fluid may be extracted directly from the environment of the wound, or can be washed off the wound using a saline buffer. The resulting solution can then be assayed for the concentration of the marker in, for example, a test tube or in a microassay plate.

Such a method will be preferable for use in cases in which the wound is too small or too inaccessible to allow access of a diagnostic device such as a dipstick. This method has the additional advantage that the wound exudate sample may be diluted.

It will be clear that an aqueous assay system is more applicable to use in a laboratory environment, whereas a diagnostic device containing the necessary reaction components will be more suitable for use in a hospital or domestic environment.

According to the present invention, the prognostic/diagnostic assay is designed so as to provide a correlation between the measured concentrations and ratios of markers of wound healing and the magnitude of response to treatment with an oxidized cellulose. Those skilled in the art will readily be able to determine concentration levels and ratios of markers which are predictive or indicative of a good response to treatment with oxidized cellulose.

Specific wound dressing materials and methods according to the present invention will now be described further with reference to the accompanying drawings, in which:

### BRIEF DESCRIPTION OF THE SEVERAL VIEWS OF THE DRAWING(S)

FIG. 1 shows measured elastase activity and combined MMP activity for 14 patients in a study;

FIG. 2 shows the measured elastase activity versus treatment time for four patients who responded well to treatment with a collagen/ORC sponge (maximum elastase activities all normalised to 100); and

FIG. 3 shows the measured elastase activity versus treatment time for four patients who did not respond well to treatment with a collagen/ORC sponge (maximum elastase activities all normalised to 100).

### DETAILED DESCRIPTION OF THE INVENTION

Preparation of the Wound Dressing Component

The collagen/ORC sponge dressing used in these studies was commercial PROMOGRAN dressing prepared substantially as described in EP-A-1153622. The following is a brief summary of the method used to make this dressing.

The collagen component is prepared from bovine corium as follows. Bovine corium is split from cow hide, scraped and soaked in sodium hypochlorite solution (0.03% w/v) to inhibit microbial activity pending further processing. The corium is then washed with water and treated with a solution containing sodium hydroxide (0.2% w/v) and hydrogen peroxide (0.02% w/v) to swell and sterilize the corium at ambient temperature. The corium splits then undergo an alkali treatment step in a solution containing sodium hydroxide, calcium hydroxide and sodium bicarbonate (0.4% w/v, 0.6% w/v and 0.05% w.v, respectively) at pH greater than 12.2, ambient temperature, and for a time of 10-14 days, with tumbling, until an amide nitrogen level less than 0.24mmol/g is reached. The corium splits then undergo an acid treatment step with 1% hydrochloric acid at ambient temperature and pH 0.8-1.2. The treatment is continued with tumbling until the corium splits have absorbed sufficient acid to reach a pH less than 2.5. The splits are then washed with water until the pH value of corium splits reaches 3.0-3.4.

The corium splits are then comminuted with ice in a bowl chopper first with a coarse comminution and then with a fine comminution setting. The resulting paste, which is made up in a ratio of 650 g of the corium splits to 100 g of water, as ice, is frozen and stored before use in the next stage of the process. However, the collagen is not freeze-dried before admixture with the ORC in the next stage.

The ORC component of the freeze-dried pad is prepared as follows. A SURGICEL cloth (Johnson & Johnson Medical, Arlington) is milled using a rotary knife cutter through a screen-plate, maintaining the temperature below 60° C. The milled ORC powder and the required weight (according to solids content) of frozen collagen paste are then added to a sufficient amount of water acidified with acetic acid to obtain a pH value of 3.0 and a total solids content of 1.0%. The mixture is homogenized through a Fryma MZ130D homogenizer, progressively diminishing the settings to form a homogeneous slurry. The pH of the slurry is maintained at 2.9-3.1. The slurry temperature is maintained below 20° C., and the solids content is maintained at 1%±0.07.

The resulting slurry is pumped to a degassing vessel. Vacuum is initiated for a minimum of 30 minutes, with intermittent stirring, to degas the slurry. The slurry is then pumped into freeze-drier trays to a depth of 25 mm. The trays are placed onto freezer shelves where the temperature has been preset to −40° C. The freeze-drier programme is then initiated to dry and dehydrothermally cross-link the collagen and ORC to form thick sponge pads.

On completion of the cycle, the vacuum is released, the freeze-dried blocks are removed, and are then split to remove the top and bottom surface layers, and to divide the remainder of the blocks into 3 mm-thick pads. The step of splitting the freeze-dried blocks into pads is carried out with a Fecken Kirfel K1 slitter.

ETH-5169USPCT Finally, the pads are die-cut to the desired size and shape on a die-cutter, packaged, and sterilized with 18-29 KGy of cobalt 60 gamma-irradiation. Surprisingly, this irradiation does not cause significant denaturation of the collagen, which appears to be stabilized by the presence of ORC. The resulting freeze-dried collagen ORC pads have a uniform, white, velvety appearance. The thickness of the pads is about 3 mm and the collagen content is about 54%.

Clinical Study and Patient Selection

All patients enrolled in this study had diabetic foot ulcers of at least 30 days duration and a surface area of at least 1 cm². Patients were excluded if the target wound showed any signs of infection or if exposed bone with positive osteomyelitis was observed. Additional exclusion criteria included concomitant conditions or treatments that may have interfered with wound healing and a history of non-compliance that would make it unlikely that a patient would complete the study. Fourteen patients meeting these study criteria were enrolled, and wound fluid collected. Informed consent was obtained from all patients or their authorised representatives prior to study enrolment and the protocol was approved by the Ethics Committee at the participating study centre prior to the commencement of the study. The study was conducted in accordance with both the Declaration of Helsinki and Good Clinical Practice.

Protein Assay

Total protein present in each extracted wound fluid sample was determined using the Bradford protein assay. The protein binding solution comprises 1 ml Coomassie Brillant Blue stock solution 200mg-Coomassie Brillant Blue G250, Sigma Chemical Co., dissolved in 50 ml ethanol-90%); 2 ml orthophosphoric acid (85% w/v); in a final volume of 20 ml with distilled water. This solution was filtered (Whatman #1 filter paper) and used immediately. The protein level in a sample wound fluid was measured by mixing 10-111 sample or standard with 190-111 of the protein binding solution in a microtitre well and incubating for 30 mins at ambient temperature prior to reading absorbance at 595 nm. The concentration of protein was estimated from a standard calibration of BSA (bovine serum albumin prepared in distilled water; Sigma Chemical Co.) ranging from 1.0 to 001 mg/ml.

Protease Activity Assays

The levels of neutrophil-derived elastase, and matrix metalloproteinases present in the wound fluid samples were measured spectrofluorimetrically using substrate activity assays. The substrates comprise short peptides synthesised to mimic the appropriate enzyme cleavage site and contain a fluorescent reporter group which is released upon hydrolysis. Enzyme activity was determined by measuring the rate of production of the fluorimetric compound, 7-amino 4-methyl coumarin. Activity was expressed either as relative fluorescence units per minute (RFU/min) or change in fluorescence when corrected for total protein (RFU/min/mg protein). Each sample was tested times 6 and the average value calculated. The substrate was prepared at 10 mM-stock concentration, and diluted to a working concentration of 0.5 mM in the appropriate assay buffer. The reaction mixture, combined in a microtitre well (black, flat bottomed) comprised 5 [symbol] l wound fluid, 175 [symbol] l assay buffer and 20 [symbol] l substrate (final concentration 50 [symbol] M). The microtitre plate was read immediately at 455 nm (excitation 383 nm) and at timed intervals over the next hour, between readings the plate was covered and incubated at 37° C.

Neutrophil-derived elastase-like activity was estimated using the fluorimetric substrate Methoxy-Alanine-Proline-Valine-7-amino 4-methyl coumarin (Bachem UK, Ltd.) solubilised in methanol. The assay buffer required for optimal activity of this enzyme was 0.1 M Hepes, pH 7.5 containing 0.5M NaCl and 10% dimethyl sulphoxide.

Matrix metalloproteinase-like activity was estimated utilising the substrate Succinyl-Glycine-Proline-Leucine-Glycine-Proline (SEQ ID NO: 26) 7-amino 4-methyl coumarin (Bachem, UK, Ltd.) solubilised in methanol. The assay buffer necessary for maximal MMP activity was 40 mM Tris/HCl, pH 7.4 containing 200 mM NaCl and 10 mM CaCl₂.

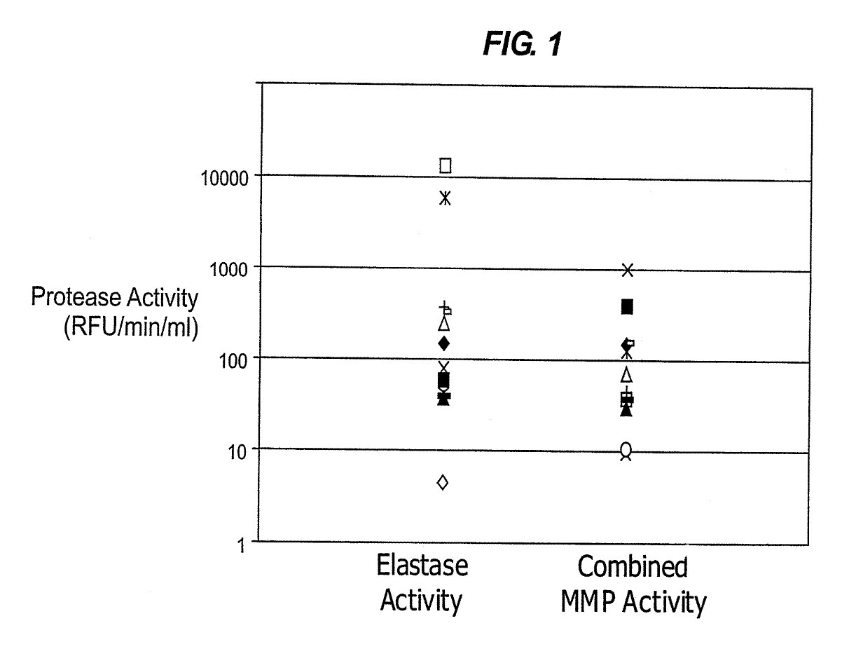

The results of the assays carried out on wound fluid samples taken from the patients immediately before treatment with the collagen/ORC dressing are shown in FIG. 1. It can be seen that the measured elastase activity ranged over three orders of magnitude, and the measured level of combined MMP activity ranged over two orders of magnitude.

Protease Inhibitor Activity Assays

AAT levels in the wound fluid was measured using a commercial ELISA kit obtained from Oxford Biosystems Ltd., Catalog Number K6750, as directed, but usually requiring a dilution factor of 1000 when measuring wound fluids.

Effect on Neutrophil Elastase Levels a/Treatment with the Collagen/ORC Dressing

Each patient was then treated by application of a PROMOGRAN dressing to the whole surface of the ulcer, together with suitable secondary dressings to hold the PROMOGRAN in place. The wound fluid from each patient was sampled at 7-day intervals, and the elastase activity was measured as described above for each sample. Patients who developed symptoms of infection, or whose treatment was discontinued for other reasons, were excluded from the study. The treatment and analysis were completed for a total of eight patients. It was found that these divided equally into a group of four who responded well to the treatment, and a group of four who responded less well to the treatment.

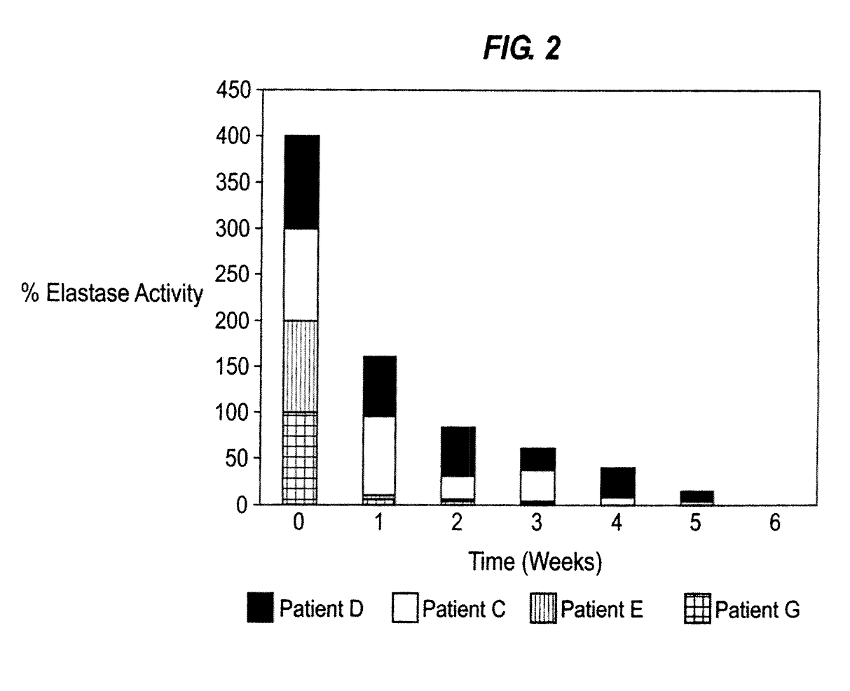

The results are shown in FIG. 2 for the group of patients who responded well to treatment with PROMOGRAN. This group was characterised by a rapid decrease in elastase activity following application of the PROMOGRAN dressing.

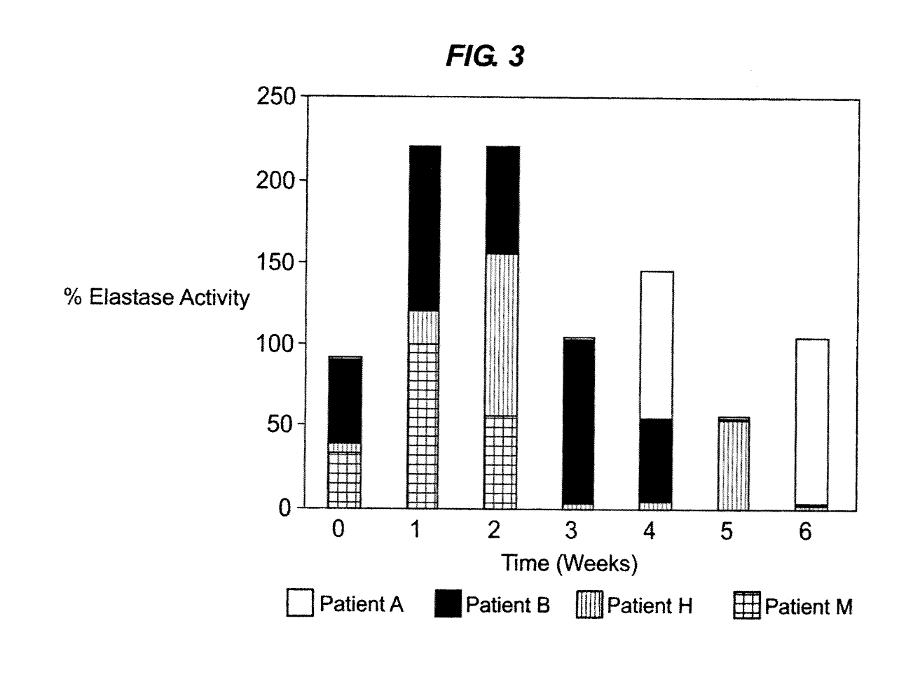

The results are shown in FIG. 3 for the group of patients who did not respond well to treatment with PROMOGRAN. This group was characterised by an increase in elastase activity following application of the PROMOGRAN dressing.

From these and other data it can be concluded that measurement of the elastase activity in wound fluid can be used to identify the patients who will benefit most and/or who are benefiting most from treatment with oxidized cellulose dressings.

Effect on AAT:Neutrophil Elastase Ratio a/Treatment with the Collagen/ORC Dressing

For comparative purposes, the AAT:elastase ratio was measured for a sample of acute wound fluid. The measured value for acute wound fluid was 221, the high value being due mainly to the very low level of neutrophil elastase in acute wound fluid.

A further group of patients with chronic venous ulcers was studied. The ratio of AAT to neutrophil elastase in wound fluid samples from each patient was determined before treatment and found to be less than 10. The drop in the ratio is mainly due to a large increase in the neutrophil elastase levels in the wound fluid. The AAT levels of some of the patients showed a relatively small decrease in the chronic wound fluid, after correction for total protein content of the wound fluid. However, some patients also exhibited a substantial drop in AAT levels, resulting in very low AAT:elastase ratios. The patients in the study then underwent treatment with PROMOGRAN for 6 weeks. The group could be divided into a sub-group of 6 patients (Group A) that exhibited better than 50% wound closure after 6 weeks of treatment, and a second sub-group of 6 patients (Group B) that exhibited less than 50% wound closure after the same period of treatment. The ratio of neutrophil elastase to AAT in wound fluid samples from each patient was determined at 3 and 6 weeks from starting treatment. The results are shown in Table 1:

TABLE 1

| Time After | | |
|---|---|---|
| Start of | Elastase:ATT Ratio | |
| Treatment | Group | Group |
| (Weeks) | A | B |
| 0 | 3.63 | 0.94 |
| 3 | 8.83 | 0.33 |
| 6 | 9.28 | 0.32 |

From these data it can be concluded that measurement of the ratio of AAT to elastase in wound fluid can be used to identify the patients who will benefit most and/or who are benefiting most from treatment with oxidized cellulose dressings. The ratio is a particularly clear and reliable diagnostic for wound chronicity, and prognostic tool for identifying chronic wounds that will benefit most from therapy with oxidized cellulose. Furthermore, the increase in the ratio over time clearly identifies those wounds that are healing as a result of the oxidized cellulose therapy, whereas the non-healing wounds exhibited a decrease in the already-low level of the ratio.

1. A diagnostic test apparatus for determining a ratio of: (a) at least one endogenous protease enzyme inhibitor, to (b) at least one endogenous protease enzyme, in a sample of a wound fluid, the diagnostic test apparatus comprising:
an indicator moiety that is immobilized or inhibited by a chemical moiety, wherein said chemical moiety comprises an exogenous peptide substrate for a protease enzyme, and said exogenous peptide substrate is cleavable by the protease enzyme to release or activate said indicator moiety.

2. The diagnostic test apparatus according to claim 1, wherein said step of determining comprises determining whether said ratio falls within a predetermined range.

3. The diagnostic test apparatus according to claim 1, wherein the said at least one protease enzyme comprises one or more proteases selected from the group consisting of neutrophil elastase, matrix metalloproteinases ( e.g. MMP-9, MMP-8, MMP-1, MMP-12), proteinase 3, plasmin, low molecular weight gelatinases and latent or active elastases, interleukin converting enzymes and tumor necrosis factor (TNFa) converting enzymes.

4. The diagnostic test apparatus according to claim 1, wherein the said at least one protease comprises one or more proteases selected from the group consisting of neutrophil elastase and matrix metalloproteinases.

5. The diagnostic test apparatus according to claim 1, wherein the said at least one protease enzyme inhibitor is selected from the group consisting of elastinil, elafin, secretory leukocyte proteinase inhibitor, alpha-1-macroglobulin, alpha-1-antitrypsin (AAT), and mixtures thereof.

6. The diagnostic test apparatus according to claim 1, wherein the said at least one protease enzyme is a neutrophil elastase, and the said at least one protease enzyme inhibitor is AAT.

7. The diagnostic test apparatus according to claim 1, wherein said apparatus comprises a single diagnostic device specifically adapted for detecting both said at least one endogenous protease enzyme and said at least one endogenous protease inhibitor in said sample.

8. The diagnostic test apparatus according to claim 1, further comprising a dip-stick or swab for sampling of said wound fluid.

9. A wound treatment system comprising the apparatus according to claim 1, and a wound dressing comprising oxidized cellulose.

10. The wound treatment system of claim 9, wherein the wound dressing comprises a combination of oxidized regenerated cellulose with collagen and/or chitosan in the dry weight ratio of from about 10:1 to about 1:10.

11. A diagnostic test apparatus for determining a ratio of: (a) at least one endogenous protease enzyme inhibitor, to (b) at least one endogenous protease enzyme, in a sample of a wound fluid, the diagnostic test apparatus comprising:
a solid support material having an immunological binding partner for an analyte moiety covalently linked thereto.

12. The diagnostic test apparatus according to claim 11, wherein said step of determining comprises determining whether said ratio falls within a predetermined range.

13. The diagnostic test apparatus according to claim 11, wherein the said at least one protease enzyme comprises one or more proteases selected from the group consisting of neutrophil elastase, matrix metalloproteinases (e.g. MMP-9, MMP-8, MMP-1, MMP-12), proteinase 3, plasmin, low molecular weight gelatinases and latent or active elastases, interleukin converting enzymes and tumor necrosis factor (TNFa) converting enzymes.

14. The diagnostic test apparatus according to claim 11, wherein the said at least one protease comprises one or more proteases selected from the group consisting of neutrophil elastase and matrix metalloproteinases.

15. The diagnostic test apparatus according to claim 11, wherein the said at least one protease enzyme inhibitor is selected from the group consisting of elastinil, elafin, secretory leukocyte proteinase inhibitor, alpha-1-macroglobulin, alpha-1-antitrypsin (AAT), and mixtures thereof.

16. The diagnostic test apparatus according to claim 11, wherein the said at least one protease enzyme is a neutrophil elastase, and the said at least one protease enzyme inhibitor is AAT.

17. The diagnostic test apparatus according to claim 11, wherein said apparatus comprises a single diagnostic device specifically adapted for detecting both said at least one endogenous protease enzyme and said at least one endogenous protease inhibitor in said sample.

18. The diagnostic test apparatus according to claim 11, further comprising a dip-stick or swab for sampling of said wound fluid.

19. A wound treatment system comprising the apparatus according to claim 11, and a wound dressing comprising oxidized cellulose.

20. The wound treatment system of claim 19, wherein the wound dressing comprises a combination of oxidized regenerated cellulose with collagen and/or chitosan in the dry weight ratio of from about 10:1 to about 1:10.


SEQUENCE LISTING (26 sequences)

SEQ ID NO: 1 - protein, 9 aa, Artificial Sequence
  Synthetic peptide
KGAAAKAAA

SEQ ID NO: 2 - protein, 4 aa, Artificial Sequence
  Synthetic peptide
AAPV

SEQ ID NO: 3 - protein, 4 aa, Artificial Sequence
  Synthetic peptide
AAPL

SEQ ID NO: 4 - protein, 4 aa, Artificial Sequence
  Synthetic peptide
AAPF

SEQ ID NO: 5 - protein, 4 aa, Artificial Sequence
  Synthetic peptide
AAPA

SEQ ID NO: 6 - protein, 4 aa, Artificial Sequence
  Synthetic peptide
AYLV

SEQ ID NO: 7 - protein, 6 aa, Artificial Sequence
  Synthetic peptide
GPXGPX

SEQ ID NO: 8 - protein, 6 aa, Artificial Sequence
  Synthetic peptide
GPLGPX

SEQ ID NO: 9 - protein, 6 aa, Artificial Sequence
  Synthetic peptide
GPIGPX

SEQ ID NO: 10 - protein, 5 aa, Artificial Sequence
  Synthetic peptide
APGLX

SEQ ID NO: 11 - protein, 7 aa, Artificial Sequence
  Synthetic peptide
PLGPXRX

SEQ ID NO: 12 - protein, 7 aa, Artificial Sequence
  Synthetic peptide
PLGLLGX

SEQ ID NO: 13 - protein, 7 aa, Artificial Sequence
  Synthetic peptide
PQGIAGW

SEQ ID NO: 14 - protein, 5 aa, Artificial Sequence
  Synthetic peptide
PLGCH

SEQ ID NO: 15 - protein, 6 aa, Artificial Sequence
  Synthetic peptide
PLGLWA

SEQ ID NO: 16 - protein, 7 aa, Artificial Sequence
  Synthetic peptide
PLALWAR

SEQ ID NO: 17 - protein, 7 aa, Artificial Sequence
  Synthetic peptide
PLAYWAR

SEQ ID NO: 18 - protein, 7 aa, Artificial Sequence
  Synthetic peptide
PLGMWSR

SEQ ID NO: 19 - protein, 4 aa, Artificial Sequence
  Synthetic peptide
GRGD

SEQ ID NO: 20 - protein, 6 aa, Artificial Sequence
  Synthetic peptide
GRGDNP

SEQ ID NO: 21 - protein, 5 aa, Artificial Sequence
  Synthetic peptide
GRGDS

SEQ ID NO: 22 - protein, 7 aa, Artificial Sequence
  Synthetic peptide
GRGDSPK

SEQ ID NO: 23 - protein, 7 aa, Artificial Sequence
  Synthetic peptide
PYAYWMR

SEQ ID NO: 24 - protein, 6 aa, Artificial Sequence
  Synthetic peptide
FRSSRQ

SEQ ID NO: 25 - protein, 9 aa, Artificial Sequence
  Synthetic peptide
MISLMKRPQ

SEQ ID NO: 26 - protein, 5 aa, Artificial Sequence
  Synthetic peptide
GPLGP